(19) United States
(12) Patent Application Publication
(10) Pub. No.: US 2007/0269365 A1
(43) Pub. Date: Nov. 22, 2007

(54) PROCESSES FOR THE PREPARATION OF ISOCYANATES

(75) Inventors: Rainer Weber, Odenthal (DE); Jurgen Kintrup, Leverkusen (DE); Andreas Bulan, Langenfeld (DE); Alfred Soppe, Issum (DE); Martin Weiss, Bornheim/Sechtem (DE)

Correspondence Address: CONNOLLY BOVE LODGE & HUTZ, LLP, P O BOX 2207, WILMINGTON, DE, 19899, US

(73) Assignee: Bayer Material Science AG, Leverkusen (DE)

(21) Appl. No.: 11/748,634

(22) Filed: May 15, 2007

(30) Foreign Application Priority Data
May 16, 2006 (DE) 102006022761.1

(51) Int. Cl. C01B 7/00; C07C 263/10

(52) U.S. Cl. 423/502; 560/347

(57) ABSTRACT

Processes are described which comprise: (a) reacting chlorine with carbon monoxide to form phosgene; (b) reacting the phosgene with at least one organic amine to form at least one isocyanate and hydrogen chloride; (c) separating the hydrogen chloride; (d) oxidizing the hydrogen chloride with oxygen in a gas phase to form additional chlorine; and (e) recycling at least a portion of the additional chlorine to the reaction of the chlorine and the carbon monoxide; wherein the oxidation of the hydrogen chloride is initiated via a high energy source which may be selected from electron-exciting radiation, ionizing radiation, a gas discharge, a plasma, and combinations thereof.

### BACKGROUND OF THE INVENTION

Chlorine is very often employed as an oxidizing agent in the production chain for the preparation of many organic compounds and for the preparation of raw materials for the production of polymers. Hydrogen chloride is often formed as a by-product in such reactions. For example, chlorine is employed in the preparation of isocyanates and hydrogen chloride is formed as a by-product. The hydrogen chloride can be utilized further, e.g., by marketing of an aqueous solution (hydrochloric acid) or by use in syntheses of other chemical products.

However, the amounts of hydrogen chloride produced as a by-product cannot always be marketed or employed for other syntheses in their entirety. Furthermore, hydrogen chloride can normally be employed for synthetic purposes only if it has been appropriately purified beforehand. The marketing and sale of by-product hydrogen chloride is usually economical only if the hydrogen chloride or the hydrochloric acid does not have to be transported over long distances. One of the more common possibilities for utilizing the by-product hydrogen chloride is use as a raw material in the preparation of PVC, in which oxychlorination of ethylene with hydrogen chloride to give ethylene dichloride takes place. Disposal of hydrogen chloride, e.g., by neutralization with alkali, can be unattractive for economic and ecological reasons.

A recycling process for the by-product hydrogen chloride and the re-introduction of the chlorine and/or the hydrogen formed by recycling into a production process from which the hydrogen chloride was obtained would therefore be desirable. Processes for producing chlorine from hydrogen chloride include oxidation of hydrogen chloride, electrolysis of gaseous hydrogen chloride and electrolysis of an aqueous solution of hydrogen chloride (hydrochloric acid). Oxidation of hydrogen chloride (HCl) to chlorine (Cl₂) takes place by reaction of hydrogen chloride and oxygen (O₂) in accordance with:
4HCl+O₂ [symbol] 2 Cl₂+2H₂O

The reaction can be carried out in the presence of catalysts. Suitable catalysts for this reaction, generally known as the Deacon reaction, are known.

The laid-open specification WO 04/14845 A1 discloses an integrated process for the preparation of isocyanates and catalytic oxidation of hydrogen chloride by the Deacon process, and the laid-open specification WO 97/24320 A1 discloses an integrated process for the preparation of isocyanates and gas phase electrolysis of hydrogen chloride.

A disadvantage of the known heterogeneously catalysed oxidation of hydrogen chloride (Deacon process) is the incomplete conversion (up to 90%) at the reaction temperature required for the reaction with the known catalysts, which is conventionally in the range of between 250 and 450° C. The product mixture of the reaction is therefore always worked up in an involved manner. A farther considerable disadvantage of the catalytic hydrogen chloride oxidation (Deacon process) is that the catalysts employed for the reaction are often exceptionally sensitive to impurities in the hydrogen chloride. The recycling capacity rapidly drops drastically as a result of a loss in the activity of the catalyst. At the same time, the working up of the reaction gas emerging from the reactor (oxygen, hydrogen chloride, chlorine, water) becomes even more involved due to the lower conversion of the hydrogen chloride oxidation in the reactor. This reduces overall the profitability of the catalytic oxidation process significantly. The hydrogen chloride containing impurities which are obtained in the preparation of isocyanates must therefore be purified in an involved manner This is normally carried out by very low temperature condensation of the impurities and/or by absorption of the impurities with active charcoal.

### BRIEF SUMMARY OF THE INVENTION

One object of the present invention includes providing a process for the preparation of isocyanates which can include at least partial recycling of the hydrogen chloride obtained during the preparation of the isocyanates, and which can be operated easily and reliably. Additionally, such processes should preferably provide stable operation of the oxidation of hydrogen chloride without prior expensive purification of the hydrogen chloride.

It has now been surprisingly found that, in contrast to the known thermally-activated, heterogeneously-catalyzed process (Deacon process), by utilizing non-thermal excitation sources, the activation energy necessary for the reaction can also be supplied without a catalyst or supply of thermal energy, it being possible for the involved prepurification of the hydrogen chloride before the oxidation to be omitted and, where appropriate, it being possible to achieve a higher yield than according to the prior art.

The invention relates, in general, to an integrated process for the preparation of isocyanates from phosgene and at least one amine, and oxidation of the hydrogen chloride thereby obtained with oxygen to give chlorine, the chlorine being at least partly recycled to the preparation of phosgene.

The invention relates, in particular, to processes for the preparation of chlorine by non-thermally activated reaction of hydrogen chloride with oxygen, in which, from the gas mixture formed during the reaction, comprising at least the target products chlorine and water, unreacted hydrogen chloride and oxygen and, where appropriate, further secondary constituents, such as carbon dioxide and nitrogen, the chlorine is removed and recycled into the preparation of phosgene.

One embodiment of a process according to the invention includes a process comprising: (a) reacting chlorine with carbon monoxide to form phosgene; (b) reacting the phosgene with at least one organic amine to form at least one isocyanate and hydrogen chloride; (c) separating the hydrogen chloride; (d) oxidizing the hydrogen chloride with oxygen in a gas phase to form additional chlorine; and (e) recycling at least a portion of the additional chlorine to the reaction of the chlorine and the carbon monoxide; wherein the oxidation of the hydrogen chloride is initiated via a high energy source.

In various preferred embodiments of processes according to the present invention, the high energy source comprises at least one selected from the group consisting of electron-exciting radiation, ionizing radiation, and/or a gas discharge which leads to the formation of a plasma, and combinations thereof.

### BRIEF DESCRIPTION OF THE SEVERAL VIEWS OF THE DRAWING

The foregoing summary, as well as the following detailed description of the invention, will be better understood when read in conjunction with the appended drawing. For the purpose of illustrating the invention, there is shown in the drawing an embodiment which is presently preferred. It should be understood, however, that the invention is not limited to the precise arrangement and instrumentalities shown.

In the drawing:

FIG. 1 is a representative flowchart of a process according to one embodiment of the present invention.

### DETAILED DESCRIPTION OF THE INVENTION

As used herein, the singular terms “a” and “the” are synonymous and used interchangeably with “one or more.” Accordingly, for example, reference to “a gas” herein or in the appended claims can refer to a single gas or more than one gas. Additionally, all numerical values, unless otherwise specifically noted, are understood to be modified by the word “about.”

The invention provides a process for the preparation of isocyanates which can include the following: (a) preparation of phosgene by reaction of chlorine with carbon monoxide; (b) reaction of the phosgene formed with at least one organic amine to form at least one isocyanate and hydrogen chloride; (c) separating off and working up of the isocyanates formed; (d) separating off of the hydrogen chloride formed; (e) oxidation of the hydrogen chloride with oxygen in the gas phase to give additional chlorine; and (f) recycling of at least some of the additional chlorine into the preparation of phosgene, characterized in that the oxidation is initiated by means of high-energy, preferably electron-exciting and/or ionizing radiation and/or a gas discharge and/or a plasma.

High-energy radiation in the context of the invention is understood as meaning radiation having an energy of at least 0.5 eV, and preferably at least 1 eV.

Preferably, the separating off of the hydrogen chloride formed in the preparation of the at least one isocyanate comprises a separating off of phosgene by liquefaction.

Processes according to various embodiments of the present invention are preferably carried out continuously, since batch or semi-batch operation, which is likewise possible, is somewhat more involved technically than continuous processes.

It has been found that at least some of the disadvantages of known Deacon processes in a combined isocyanate preparation process can be overcome in particular if the reaction of hydrogen chloride with oxygen is carried out at a pressure of for example I bar, and at a temperature of, in particular, below 250° C., preferably below 200° C., and particularly preferably not more than 150° C. At temperatures of below 250° C., the thermodynamically possible equilibrium conversion for the reaction:
4 HCl+O₂ [symbol] 2 Cl₂+2 H₂O
increases as the temperature decreases under constant pressure.

The ratio of O₂ to HCl can be 1:4 to 10:1 and the pressure can be 0.1 to 10 bar. Preferably the temperature and pressure are selected such that the condensation of water or aqueous hydrochloric acid does not take place.

Processes in which chemical reactions are activated non-thermally are described, for example, in W. Stiller, Nichtthermische aktivierte Chemie, Birkhäuser Verlag, Basle, Boston, 1987, p. 33-34, p. 45-49, p. 122-124, p. 138-145, the entire contents of which are incorporated herein by reference. Non-thermally activated reactions are understood as meaning, for example, excitations of the reaction using any of the following:
high-energy radiation, e.g., laser radiation, photochemical radiation sources, UV radiation, infra-red radiation and the like;
a low temperature plasma, e.g., generated by electrical discharge;
magnetic field excitation;
tribomechanical activation, e.g., excitation by shock waves;
ionizing radiation, e.g., gamma and x-ray radiation, α- and β-rays from nuclear decays, high-energy electrons, protons, neutrons and heavy ions;
microwave irradiation; and/or
electromagnetic radiation in the radiofrequency range.

Non-thermally activated hydrogen chloride oxidation processes are described, for example, in the specifications: JP 59073405, RU-A 2253607, DD 88 309, SU-A 1801943, I&EC Fundamentals 7(3), 400-409 (1968), the entire contents of each of which are incorporated herein by reference.

JP 59073405 describes the photo-oxidation of gaseous hydrogen chloride under pressures of between 0.5 and 10 atm and at temperatures of from 0 to 400° C., pulsed laser radiation (3×10⁻¹⁵ s pulse duration and 0.01-100 J energy, e.g., KrF laser (wavelength 249 nm, 10 W output)) or a high voltage mercury lamp (100 W output) or also a combination of the two sources of radiation mentioned being employed, inter alia, for excitation of the reactants. The non-thermal excitation takes place by UV radiation with both sources of radiation. Such a process is suitable for use in various embodiments of the present invention for the oxidation of hydrogen chloride.

RU-A 2253607 describes a process, carried out at 25 to 30° C., for the preparation of chlorine in which a gaseous hydrogen chloride-air mixture flows at a speed of 1 to 30 m/s through a tube reactor and the activation of the reactants takes place in a reaction zone by a mercury vapour lamp with a volumetric radiation density in the range of from 10×10⁻⁴ to 40×10⁻⁴ W/cm³ and under a pressure of 0.1 MPa. Such a process can preferably be used in various embodiments of the present invention.

In various preferred embodiments of processes according to the present invention, the oxidation of hydrogen chloride with oxygen is initiated by at least one radiation type from the series: UV radiation, in particular in the wavelength range of from 50 to 300 nm, x-ray radiation, gamma radiation, synchrotron radiation, electron radiation, neutron radiation, heavy ion radiation or alpha radiation.

A process in which UV radiation which is generated by a low pressure mercury vapour lamp, medium pressure mercury vapour lamp, high pressure mercury vapour lamp, a UV laser, in particular an excimer laser, and/or frequency-multiplied IR laser is used as the high energy source is particularly preferred.

Mercury vapour lamps emit radiation in various wavelength ranges, depending on the filling pressure, and this is understood to be known by those of ordinary skill in the art. For example, low pressure mercury vapour lamps typically operate under a pressure of 150 Pa and emit radiation in the range of 185 nm and 254 nm, that is to say predominantly in the UV range, and are therefore particularly suitable for initiation of the hydrogen chloride oxidation.

Further examples of sources of UV radiation are medium pressure mercury vapour lamps and high pressure mercury vapour lamps. Depending on the pressure, these lamps emit with a partial loss of the short wavelength UVC radiation (<280 nm), compared with the low pressure lamps.

Where by laser radiation is used, both pulsed and continuous laser radiation can be used.

Oxidation of hydrogen chloride in accordance with the present invention may also include the use of an oxidation catalyst, at a temperature of 150°-250° C., and oxidation initiation with a high energy source, e.g., UV radiation.

In various preferred embodiments of processes according to the present invention, a high frequency/microwave plasma, in particular having an excitation in the frequency range of from 10⁶ Hz to 10¹² Hz, is used as the plasma for initiation of the oxidation of hydrogen chloride with oxygen.

An example of a further method which can be used for initiation of the oxidation reaction with a high-energy plasma is described in the specification SU-A-1801943.

The publication I&EC Fundamentals 7(3), p. 400-409 (1968) describes in principle the excitation of the hydrogen chloride oxidation reaction with microwaves, which can particularly preferably be employed in various embodiments of the processes according to the present invention.

In the case of excitation with high-energy electrons, these can be generated, inter alia, by electrodeless discharge, thermionic discharge, glow discharge, electrical pulse discharge or other types familiar to the person skilled in the art and can be obtained by acceleration through an electrical field. In this context, the excitation can take place continuously or in pulsed form. While not bound by any particular theory, it is generally believed that the excitation of the reactants can then take place by collision with the accelerated, high-energy electrons, wherein the energy of the electrons rather than the nature of the generation of the electrons has influence on the collision excitation.

A process in which a silent spark discharge, an electrical pulse discharge, a hollow cathode discharge, a glow discharge, corona discharge or a barrier discharge is used as a gas discharge for initiation of the oxidation of hydrogen chloride with oxygen is also particularly preferred.

In addition to methods for plasma generation which are based on excitation with electrostatic fields, electromagnetic fields can also be used for generation of plasma, for example strong electromagnetic alternating fields on two capacitor plates or an inductive (electrodeless) electromagnetic excitation, in which alternating current is passed through an excitation coil and an electrical field is thereby induced in the gas space, the field generating the charge carriers in the gas space. A further form of electromagnetic excitation lies in excitation by microwave radiation, in which microwave radiation is passed into the reaction space through a suitable hollow conductor geometry.

The energy carriers in a non-thermal excitation can be fed both into a gas mixture of hydrogen chloride and oxygen (educts) and to the individual educts. It is also possible to excite only one reactant and to feed in the other reactants downstream. Non-thermal excitation of the educt mixture is preferably carried out.

In various preferred embodiments of processes according to the present invention, oxygen having a purity of at least 93 vol. %, in particular of at least 99 vol. %, is used for the oxidation reaction. The formation of nitrogen oxides, which may arise, e.g., in the HCl oxidation processes known from the specification SU-A-1801943 or RU-A 2253607, can be largely avoided in this way. Nitrogen oxides are undesirable in the overall process, in particular, since they are generally corrosive harmful gases and in some cases are difficult to separate off from chlorine. Since the chlorine is recycled, the nitrogen oxides can cause damage in the installations for carrying out the initial reactions.

As described above, the process according to the invention includes an integrated process for the preparation of isocyanates and the oxidation of hydrogen chloride for recovery of chlorine for the synthesis of phosgene as a starting substance for the preparation of isocyanates.

In a first step of a preferred process according to various embodiments of the present invention, which provides the integration of the chlorine preparation process into a preparation of isocyanates, the preparation of phosgene is carried out by reaction of chlorine with carbon monoxide. The synthesis of phosgene is adequately known and is described, e.g., in Ullmanns Enzylopädie der industriellen Chemie, 3rd edition, volume 13, page 494-500, the entire contents of which are incorporated herein by reference. Further processes for the preparation of isocyanates are described, e.g., in U.S. Pat. No. 4,764,308 and WO 03/072237, the entire contents of each of which are incorporated herein by reference. On an industrial scale, phosgene is predominantly prepared by reaction of carbon monoxide with chlorine, preferably on active charcoal as a catalyst. The highly exothermic gas phase reaction is carried out at temperatures of up to 400° C., normally in tube bundle reactors, the product conventionally being obtained at 40-150° C. The heat of reaction can be removed in various ways, for example by a liquid heat exchange medium, as described e.g., in WO 03/072237, or by evaporative cooling via a secondary cooling circulation, the heat of reaction simultaneously being utilized for generation of steam, as disclosed e.g., in U.S. Pat. No. 4,764,308.

At least one isocyanate is then formed from the phosgene by reaction with at least one organic amine or a mixture of two or more amines in a subsequent step. This step is also referred to as phosgenation herein. The phosgenation also results in the formation of hydrogen chloride as a by-product.

The synthesis of isocyanates is likewise adequately known from the prior art, as a rule phosgene being employed in a stoichiometric excess, based on the amine. The phosgenation according to b) conventionally takes place in the liquid phase, it being possible for the phosgene and the amine to be dissolved in a solvent. Preferred solvents are chlorinated aromatic hydrocarbons, such as, for example, chlorobenzene, o-dichlorobenzene, p-dichlorobenzene, trichlorobenzenes, the corresponding chlorotoluenes or chloroxylenes, chloroethylbenzene, monochlorodiphenyl, α- and β-naphthyl chloride, ethyl benzoate, dialkyl phthalates, diisodiethyl phthalate, toluene and xylenes. Further examples of suitable solvents are known from the prior art. As is moreover known from the prior art, e.g., WO 96/16028, the entire contents of which are incorporated herein by reference, the isocyanate formed can likewise itself function as a solvent for phosgene. In another preferred embodiment, the phosgenation, in particular of suitable aromatic and aliphatic diamines, takes place in the gas phase, i.e., above the boiling point of the amine. Gas phase phosgenation is described, e.g., in EP 570 799 A, the entire contents of which are incorporated herein by reference. Advantages of this process over the otherwise conventional liquid phase phosgenation lie in the saving of energy due to the minimizing of an involved solvent and phosgene circulation.

Suitable organic amines are in principle all primary amines having one or more primary amino groups which can react with phosgene to form one or more isocyanates having one or more isocyanate groups. The amines contain at least one, preferably two, or optionally three and more primary amino groups. Thus, possible organic primary amines are aliphatic, cycloaliphatic, aliphatic-aromatic and aromatic amines, di- and/or polyamines, such as aniline, halogen-substituted phenylamines, e.g., 4-chlorophenylamine, 1,6-diaminohexane, 1-amino-3,3,5-trimethyl-5-amino-cyclohexane, 2,4- or 2,6-diaminotoluene or mixtures thereof, 4,4′-, 2,4′- or 2,2′-diphenylmethanediamine or mixtures thereof, and also higher molecular weight isomeric, oligomeric or polymeric derivatives of the said amines and polyamines. Further possible amines are known from the prior art. Preferred amines for the present invention are the amines of the diphenylmethanediamine series (monomeric, oligomeric and polymeric amines), 2,4- and 2,6-diaminotoluene, isophoronediamine and hexamethylenediamine. The corresponding isocyanates diisocyanatodiphenylmethane (MDI, monomeric, oligomeric and polymeric derivatives), toluylene-diisocyanate (TDI), hexamethylene-diisocyanate (HDI) and isophorone-diisocyanate (IPDI) are obtained in the phosgenation.

The amines can be reacted with phosgene in a one-stage or two-stage or optionally multi-stage reaction. In this context, a continuous and also discontinuous mode of operation is possible.

If a one-stage phosgenation in the gas phase is chosen, the reaction is carried out above the boiling temperature of the amine, preferably within an average contact time of from 0.5 to 5 seconds and at temperatures of from 200 to 600° C., optionally while also injecting nitrogen.

In the phosgenation in the liquid phase, temperatures of 20 to 240° C. and pressures of from 1 to approx. 50 bar are conventionally employed. The phosgenation in the liquid phase can be carried out in one stage or several stages, it being possible for phosgene to be employed in a stoichiometric excess. In this context, the amine solution and the phosgene solution are combined via a static mixing element and then passed, for example, from the bottom upwards through one or more reaction towers, where the mixture reacts to give the desired isocyanate. In addition to reaction towers which are provided with suitable mixing elements, it is also possible to employ reaction tanks with a stirring device. In addition to static mixing elements, specific dynamic mixing elements can also be used. Suitable static and dynamic mixing elements are known from the prior art.

Normally, the continuous liquid phase preparation of isocyanates is carried out on an industrial scale in two stages. In this context, in the first stage in general at temperatures of not more than 220° C., preferably not more than 160° C., the carbamoyl chloride is formed from the amine and phosgene and the amine hydrochloride is formed from the amine and the hydrogen chloride split off. This first stage is highly exothermic. In the second stage, both the carbamoyl chloride is cleaved into isocyanate and hydrogen chloride and the amine hydrochloride is converted into the carbamoyl chloride. The second stage is as a rule carried out at temperatures of at least 90° C., preferably of from 100 to 240° C.

After the phosgenation, the isocyanates formed in the phosgenation are separated off. This can be effected by first separating the reaction mixture of the phosgenation into a liquid and a gaseous product stream in a manner known to the person skilled in the art. The liquid product stream substantially contains the isocyanate or isocyanate mixture, the solvent and a small portion of unreacted phosgene. The gaseous product stream substantially comprises hydrogen chloride gas, stoichiometrically excess phosgene and small amounts of solvent and inert gases such as, for example, nitrogen and carbon monoxide. The liquid stream can then subsequently be fed to a working up, preferably a working up by distillation, phosgene and the solvent being separated off in succession. If appropriate, a further working up of the isocyanates formed is moreover carried out. This can be effected, for example, by fractionating the isocyanate product obtained in a manner known to the person skilled in the art.

Subsequently, the hydrogen chloride produced during the phosgenation can be separated off from the gaseous product stream. The gaseous product stream which is obtained in the separating off of the isocyanate can be treated such that the phosgene can be fed back to the phosgenation and the hydrogen chloride can be fed to the oxidation with oxygen.

The separating off of the hydrogen chloride is generally carried out first by separating off phosgene from the gaseous product stream. The phosgene can be separated off by liquefying the phosgene, for example on one or more condensers connected in series. The liquefaction is preferably carried out at temperatures in the range of from −15 to −40°, depending on the solvent employed Solvent residues are moreover removed from the gaseous product stream by this deep-freezing.

Additionally or alternatively, the phosgene can be washed out of the gas stream in one or more stages with a cold solvent or solvent-phosgene mixture. Suitable solvents for this are, for example, the solvents chlorobenzene and o-dichlorobenzene already employed in the phosgenation. The temperature of the solvent or solvent-phosgene mixture for this is in the range of from −15 to −46° C.

The phosgene separated off from the gaseous product stream can be fed back to the phosgenation reaction. The hydrogen chloride obtained after separating off of the phosgene and a portion of the residual solvent can also contain, in addition to the inert gases, such as nitrogen and carbon monoxide, 0.1 to 1 wt. % of solvent and 0.1 to 2 wt. % of phosgene.

If appropriate, a purification of the hydrogen chloride can be carried out in order to reduce the solvent content. This can be carried out, for example, by freezing out, by passing the hydrogen chloride, for example, through one or more cold traps, depending on the physical properties of the solvent.

At least some of the additional chlorine prepared by oxidation of the hydrogen chloride is recycled into the preparation of phosgene. Before the recycling, the chlorine is preferably fed to a condensation unit in order to separate off condensable contents, such as water, hydrochloric acid and solvent residues. The condensation unit can comprise, for example, one or more cooling stages downstream, for example one or more tubular heat exchangers. Contents of hydrogen chloride in the chlorine can also be absorbed in dilute hydrochloric acid or water.

The chlorine can then be dried. The drying can be carried out, for example, with the aid of a suitable drying agent in an absorption column equipped with mass transfer elements. A suitable drying agent can be as described in DE 10 235 476 A, the entire contents of which are incorporated herein by reference, in addition to molecular sieves or hygroscopic adsorbents, e.g., sulfuric acid. The drying can be carried out in one or more stages. The drying is preferably carried out in two stages, by bringing the chlorine to be dried into contact with a sulfuric acid of relatively low concentration, preferably 70 to 80%, particularly preferably 75 to 80%, in a first stage. In a second stage, the residual moisture is removed from the chlorine by means of a more highly concentrated sulfuric acid of preferably 88 to 96%, particularly preferably 92-96%. The chlorine dried in this manner and having a residual moisture content of preferably not more than 100 ppm, particularly preferably not more than 20 ppm, can be passed through a demister in order to remove any droplets of sulfuric acid still contained therein.

The circulation procedure of a process according to the invention can require, where appropriate, a further part amount of chlorine to be provided for the phosgene preparation in addition to the additional chlorine prepared by oxidation, since losses of chlorine and hydrogen chloride may occur in the chlorine-hydrogen chloride circulation. The provision of a further part amount of chlorine can originate in the form of elemental chlorine from an external source, for example the electrolysis of an aqueous sodium hydroxide solution or hydrochloric acid.

If a missing amount is replaced by external source chlorine, this chlorine, which can be prepared, for example, by hydrolysis of rock salt, may contain small amount of bromine or iodine. If this chlorine is employed for the preparation of MDI, discolouration of the polyurethane products prepared from MDI may occur at a certain concentration of bromine compounds and iodine compounds, as described e.g., in DE 10 235 476 A. The chlorine recycled by the process according to the invention, on the other hand, is largely bromine- and iodine-free, so that a certain bromine and iodine content is established, according to the ratio of the chlorine fed in from the outside to the recycled chlorine. A preferred embodiment of the process according to the invention accordingly comprises employing the further part amount of chlorine fed in from the outside in the preparation of phosgene for TDA phosgenation, while the low-bromine and -iodine chlorine from the oxidation according to the invention is utilized in the preparation of phosgene for the phosgenation of MDA (diphenylmethanediarmine). Bromine and iodine are bonded in the TDI in the preparation of TDI by phosgenation of TDA, and are therefore withdrawn from the hydrogen chloride circulation. In the working up of TDI by distillation, however, bromine and iodine are separated from the TDI and remain in the residue.

In a further preferred embodiment of a process according to the invention, the carbon monoxide employed in the preparation of phosgene can be prepared by reaction of methane with water or optionally with carbon dioxide in a steam reformer, and the hydrogen obtained in this procedure can be reacted with at least one organic nitro compound to give at least one amine, which is used in the preparation of the isocyanate. The preparation of carbon monoxide by reaction of methane with water in a steam reformer has been known for a long time. The reaction of hydrogen with an organic dinitro compound for the preparation of an amine (hydrogenation) is likewise known. If a steam reformer is employed for the preparation of carbon monoxide, the stoichiometrically required amount of carbon monoxide for the phosgene preparation and the stoichiometric amount of water for the hydrogenation of the dinitro compounds are available. Nitro compounds which can be employed are, for example, nitrobenzene and dinitrotoluene (DNT). Nitrobenzene and dinitrotoluene are hydrogenated to aniline and toluyenediamine (TDA). Aniline is further processed to polyamines of the diphenylmethane series. In addition to other amines, MDA and TDA can be employed for the preparation of isocyanates according to step b). In the consideration of the profitability of the overall process for the preparation of isocyanates, the preparation of carbon monoxide is also included, the carbon monoxide preferably being prepared from natural gas in a steam reformer. If other reformer processes are used, e.g., gasification of coal or cracking of petroleum fractions, other ratios of carbon monoxide to hydrogen are obtained. The higher the ratio of carbon monoxide to hydrogen, the less economical the overall process, since the missing hydrogen for the hydrogenation of the dinitro compound to give the homologous diamines must be supplied from a further source. The missing hydrogen can be provided, for example, by the electrolysis of sodium chloride or hydrochloric acid.

The advantages of an integrated process according to the invention for the preparation of isocyanates, including oxidation of the hydrogen chloride obtained in the preparation of the isocyanates to recover chlorine for the synthesis of phosgene, include the result that an involved prepurification of the hydrogen chloride can be omitted, in contrast to known thermal catalysed processes (e.g., Deacon process).

The isocyanates from the process according to the invention can be used in a conventional manner, e.g., for the preparation of plastics, lacquers, adhesives and/or sealants.

The following examples are for reference and do not limit the invention described herein.

### EXAMPLES

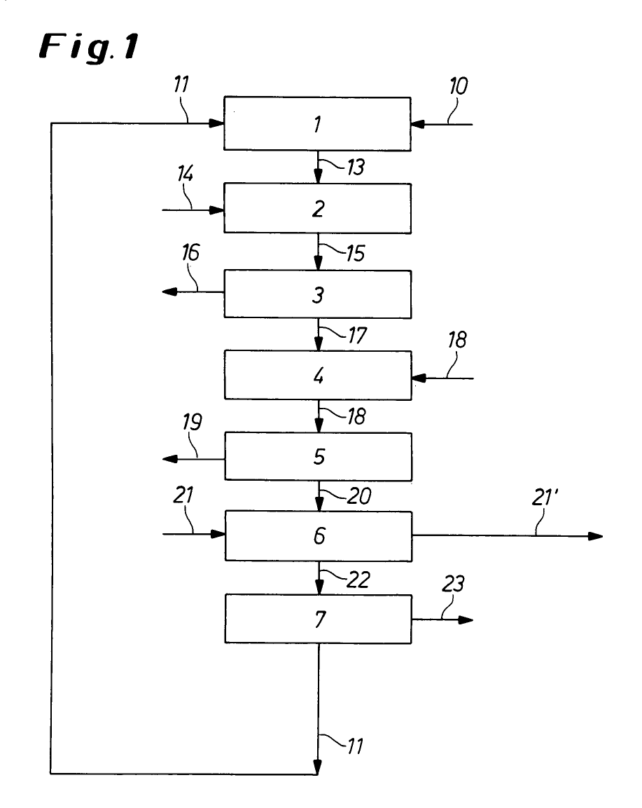

A process according to an embodiment of the invention is explained in more detail with reference to FIG. 1. FIG. 1 is a representative flowchart depicting a process according to the invention for the preparation of TDI.

In a first stage 1 of the preparation of isocyanates, chlorine 11 is reacted with carbon monoxide 10 to give phosgene 13. In the following stage 2, phosgene 13 from stage 1 is reacted with an amine 14 (in this case toluenediamine) to give a mixture 15 of isocyanate (toluene-diisocyanate, TDI) and hydrogen chloride, the isocyanate 16 is separated off (in stage 3) and utilized. The HCl gas 17 is reacted with oxygen 18 in the HCl oxidation process 4.

For this e.g., a UV-transparent reaction tube can be used, this being charged with HCl and O₂ in the stoichiometric ratio of 4:1. The reaction mixture is irradiated in the reaction tube with short wavelength (<250 nm), coherent UV light with the aid of a pulsed excimer laser. The temperature of the reaction mixture is kept at 200° C. by suitable heat exchangers during this procedure.

The reaction mixture formed from stage 4 is cooled (step 5). Aqueous hydrochloric acid 19 which is obtained by this procedure and, where appropriate, is mixed with water or dilute hydrochloric acid is sluiced out of the system.

The gas mixture 20 obtained in this way from stage 5, comprising at least chlorine, oxygen and, where appropriate, secondary constituents, such as nitrogen, carbon dioxide etc., is dried with conc. sulfuric acid 21 (96% strength) (step 6).

In a purification stage 7, chlorine 11 is separated from the dried gas mixture 21 from stage 6. The residual stream 23 containing oxygen and, where appropriate, secondary constituents is recycled, where appropriate, into the oxidation 4.

The chlorine gas 11 obtained from the purification stage 7 is employed again directly in the phosgene synthesis 1.

It will be appreciated by those skilled in the art that changes could be made to the embodiments described above without departing from the broad inventive concept thereof: It is understood, therefore, that this invention is not limited to the particular embodiments disclosed, but it is intended to cover modifications within the spirit and scope of the present invention as defined by the appended claims

What is claimed is:

1. A process comprising.
(a) reacting chlorine with carbon monoxide to form phosgene;
(b) reacting the phosgene with at least one organic amine to form at least one isocyanate and hydrogen chloride;
(c) separating the hydrogen chloride;
(d) oxidizing the hydrogen chloride with oxygen in a gas phase to form additional chlorine; and
(e) recycling at least a portion of the additional chlorine to the reaction of the chlorine and the carbon monoxide;
wherein the oxidation of the hydrogen chloride is initiated via a high energy source.

2. The process according to claim 1, wherein the high energy source comprises at least one selected from the group consisting of electron-exciting radiation, ionizing radiation, a plasma-forming gas discharge, a plasma, and combinations thereof.

3. The process according to claim 1, wherein separation of the hydrogen chloride comprises phosgene liquefaction.

4. The process according to claim 1, wherein the hydrogen chloride and the oxygen are mixed prior to oxidation initiation.

5. The process according to claim 1, wherein the high energy source comprises a gas discharge selected from the group consisting of a silent spark discharge, an electrical pulse discharge, a hollow cathode discharge, a glow discharge, a barrier discharge and combinations thereof.

6. The process according to claim 1, wherein the high energy source comprises radiation selected from the group consisting of UV radiation, x-ray radiation, gamma radiation, synchrotron radiation, electron radiation, neutron radiation, heavy ion radiation, alpha radiation or combinations thereof.

7. The process according to claim 1, wherein the high energy source comprises IN radiation generated by a light source selected from the group consisting of low pressure mercury vapour lamps, medium pressure mercury vapour lamps, high pressure mercury vapour lamps, UV lasers, frequency-multiplied IR lasers, and combinations thereof.

8. The process according to claim 1, wherein the high energy source comprises UV radiation having a wavelength of 50 nm to 300 nm.

9. The process according to claim 1, wherein the high energy source comprises a plasma selected from the group consisting of a microwave plasma having an excitation frequency of from 0.3 GHz to 300 GHz, and a high frequency plasma having an excitation frequency of from 10⁶ Hz to 10¹² Hz.

10. The process according to claim 1, wherein the oxidation of the hydrogen chloride is carried out at a temperature of 250° C. or less.

11. The process according to claim 5, wherein the oxidation of the hydrogen chloride is carried out at a temperature of 250° C. or less.

12. The process according to claim 6, wherein the oxidation of the hydrogen chloride is carried out at a temperature of 250° C. or less.

13. The process according to claim 9, wherein the oxidation of the hydrogen chloride is carried out at a temperature of 250° C. or less.

14. The process according to claim 1, wherein the oxidation of the hydrogen chloride is carried out at a temperature of 200° C. or less.

15. The process according to claim 1, wherein the oxidation of the hydrogen chloride is carried out at a temperature of 150° C. or less.

16. The process according to claim 1, wherein the oxygen has a purity of at least 93 vol. %.

17. The process according to claim 1, further comprising reacting methane and water in a steam reformer to form hydrogen and at least a portion of the carbon monoxide, and reacting the hydrogen with at least one organic nitro compound to form at least a portion of the at least one organic amine.

18. The process according to claim 1, wherein the chlorine further comprises an additional halogen selected from the group consisting of bromine, iodine and mixtures thereof.
